(19) United States
(12) Patent Application Publication
(10) Pub. No.: US 2024/0017010 A1
(43) Pub. Date: Jan. 18, 2024

(54) INJECTOR APPARATUS FOR TWO-PART INJECTANT

(71) Applicant: Grand Valley State University, Allendale, MI (US)

(72) Inventors: Robert Marshall Werner, JR., Grand Rapids, MI (US); Kelly E. Martin, Oakland Township, MI (US); Christopher P. Pung, Portland, MI (US)

(73) Assignee: Grand Valley State University, Allendale, MI (US)

(21) Appl. No.: 18/221,591

(22) Filed: Jul. 13, 2023

Related U.S. Application Data
(60) Provisional application No. 63/389,485, filed on Jul. 15, 2022.

(51) Int. Cl. A61M 5/24; A61M 5/315; A61M 5/32

(52) U.S. Cl. CPC A61M 5/2448; A61M 5/31513; A61M 5/32

(57) ABSTRACT

An injection apparatus for administering an injectant includes a mixing chamber having an impermeable membrane disposed therein, a first chamber containing a dry component, and a second chamber containing a liquid component. The dry component and the liquid component combine to form a liquid injection solution in the mixing chamber. The mixing chamber is configured to retain the liquid injection solution formed by a combination of the dry component and the liquid component. The injection apparatus is selectively configured in a plurality of states including a storage state and a mixing state.

### CROSS-REFERENCE TO RELATED APPLICATIONS

This application claims priority under 35 U.S.C. § 119(e) and the benefit of U.S. Provisional Application No. 63/389,485 entitled INJECTOR APPARATUS FOR TWO-PART INJECTANT, filed on Jul. 15, 2022, by Robert Marshall Werner Jr., et al., the entire disclosure of which is incorporated herein by reference.

### TECHNOLOGICAL FIELD

The present disclosure generally relates to an injection apparatus and, more specifically, relates to a multi-stage mixing and injection apparatus.

### SUMMARY OF THE INVENTION

In one aspect, an injection apparatus for administering an injectant includes a mixing chamber having an impermeable membrane disposed therein, a first chamber containing a dry component, and a second chamber containing a liquid component. The dry component and the liquid component combine to form a liquid injection solution in the mixing chamber. The mixing chamber is configured to retain the liquid injection solution formed by a combination of the dry component and the liquid component. The injection apparatus is selectively configured in a plurality of states comprising a storage state and a mixing state. The dry component and the liquid component are separated by the impermeable membrane in the storage state and merged in the mixing state, and the dry component is disposed within the mixing chamber in the storage state. At least one injection needle is in connection with the mixing chamber. The injection needle is placed in fluid communication with the liquid injection solution in response to a rupture of the impermeable membrane.

In another aspect, the disclosure provides for a method for administering an injectant that includes providing a dry component in a first chamber and providing a liquid component in a second chamber. A first seal between the first chamber and the second chamber is ruptured and the dry component and the liquid component are mixed in a third chamber forming the injectant by a combination of the dry component and the liquid component. The injectant is delivered to an injector assembly comprising at least one injection needle via an outlet in connection with the third chamber and administered via the at least one injection needle by applying pressure to the third chamber.

In yet another aspect, an injection apparatus is disclosure that is configured to administer an injectant. The injection apparatus includes a first chamber containing a dry component, a second chamber containing a liquid component, and a first frangible seal disposed between the first chamber and the second chamber. A third chamber is formed by a union of the first chamber and the second chamber. A fluid outlet port is in fluid communication with at least one injection needle. A second frangible seal is disposed between the third chamber and the fluid outlet port. The first frangible seal is selectively ruptured allowing the dry component to dissolve in the liquid component forming the injectant. The second frangible seal is selectively ruptured thereby placing the third chamber in fluid communication with the at least one injection needle.

These and other features, advantages, and objects of the present device will be further understood and appreciated by those skilled in the art upon studying the following specification, claims, and appended drawings.

### BRIEF DESCRIPTION OF THE DRAWINGS

The invention will now be described with reference to the following drawings, in which:

FIG. 1A is a side view of an injection apparatus;

FIG. 1B is a side profile view of an injection apparatus with a housing hidden to show detail;

FIG. 2 is an exploded assembly view of an injection apparatus;

FIG. 3A is a first schematic representation of a sequential actuation, mixing, and injection procedure for an injection apparatus;

FIG. 3B is a second schematic representation of a sequential actuation, mixing, and injection procedure for an injection apparatus;

FIG. 3C is a third schematic representation of a sequential actuation, mixing, and injection procedure for an injection apparatus;

FIG. 3D is a fourth schematic representation of a sequential actuation, mixing, and injection procedure for an injection apparatus;

FIG. 3E is a fifth schematic representation of a sequential actuation, mixing, and injection procedure for an injection apparatus;

FIG. 4A is a perspective view of an injection apparatus in a first configuration or storage configuration;

FIG. 4B is a perspective view of an injection apparatus in a second configuration or mixing configuration;

FIG. 4C is a perspective view of an injection apparatus in a third configuration or an injection configuration;

FIG. 4D is a schematic diagram of the injection apparatus demonstrated in a storage configuration;

FIG. 4E is a schematic diagram of the injection apparatus demonstrated in a mixing configuration;

FIG. 4F is a schematic diagram of the injection apparatus demonstrated in an injection configuration;

FIG. 5A is a simplified cross-sectional view of an injection apparatus in the form of a patch device in a resting configuration;

FIG. 5B is a simplified cross-sectional view of the injection apparatus in the form of a patch device in a mixing configuration;

FIG. 5C is a simplified cross-sectional view of the injection apparatus in the form of a patch device in an application or injection configuration;

FIG. 5D is a simplified top view of the injection apparatus demonstrated in FIGS. 5A-5C;

FIG. 6A is a diagram of an injection apparatus comprising a storage and mixing vessel and an injection attachment;

FIG. 6B is a schematic diagram of the injection apparatus demonstrated in shipping configuration;

FIG. 6C is a schematic diagram of the injection apparatus demonstrated in a charged or storage configuration;

FIG. 6D is a schematic diagram of the injection apparatus demonstrated in a mixing configuration;

FIG. 6E is a schematic diagram of the injection apparatus demonstrated in primed configuration; and

FIG. 6F is a schematic diagram of the injection apparatus demonstrated in an injection configuration.

### DETAILED DESCRIPTION OF EMBODIMENTS

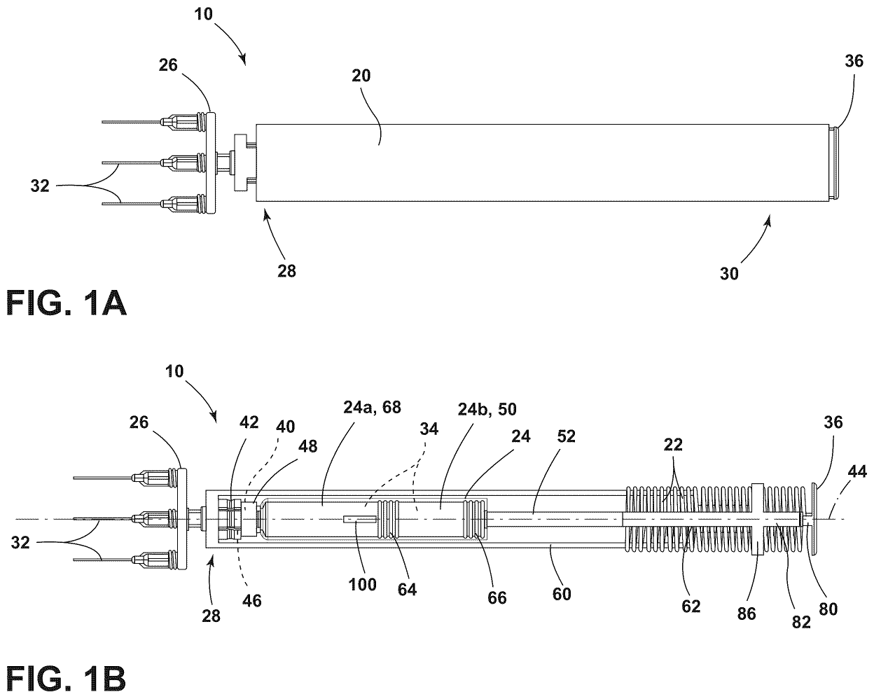

For purposes of description herein, the terms “upper,” “lower,” “right,” “left,” “rear,” “front,” “vertical,” “horizontal,” and derivatives thereof shall relate to the invention as oriented in FIG. 1. Unless stated otherwise, the term “front” shall refer to the surface of the element closer to an intended viewer of the display mirror, and the term “rear” shall refer to the surface of the element further from the intended viewer of the display mirror. However, it is to be understood that the invention may assume various alternative orientations, except where expressly specified to the contrary. It is also to be understood that the specific devices and processes illustrated in the attached drawings and described in the following specification are simply exemplary embodiments of the inventive concepts defined in the appended claims. Hence, specific dimensions and other physical characteristics relating to the embodiments disclosed herein are not to be considered as limiting, unless the claims expressly state otherwise.

The terms “including,” “comprises,” “comprising,” or any other variation thereof, are intended to cover a non-exclusive inclusion, such that a process, method, article, or apparatus that comprises a list of elements does not include only those elements but may include other elements not expressly listed or inherent to such process, method, article, or apparatus. An element preceded by “comprises a . . . ” does not, without more constraints, preclude the existence of additional identical elements in the process, method, article, or apparatus that comprises the element.

The disclosure generally relates to an injection apparatus that is configured to store a compound injectant composed of a liquid component and a dry or powder component. In various implementations, the injectant may correspond to a medication or substance for injection into a living body. In some cases, the injectant may correspond to an anti-venom, antidote, allergy treatment, neutralizing or stabilizing agent, synthetic hormone (e.g., epinephrine), or various chemicals that may be administered through one or more sites via subcutaneous or intermuscular injection. In general, the injectant may be utilized as a countermeasure response to an allergic reaction or reaction to any acute exposure, sting, or envenomation. For example, the injectant may be administered in response to exposure to stinging vegetation (e.g., barbs, nettles, etc.), snake bites, insect bites (e.g., scorpion stings, bee stings, etc.), arachnid bites, etc. Accordingly, the injectant may provide for the treatment of exposure to various neurotoxins, cytotoxins, acids, histamines, biogenic amines, or other toxins or chemicals that may be treated by injection. Though specifically discussed in reference to an antidote for venom, stings, or allergen exposure, the chemicals or components forming the injectant, may vary broadly as shall be understood by those skilled in the art. Accordingly, the injection apparatus may be implemented for any suitable injectant and may be particularly useful for administering injectants composed of a dry component and a liquid component as provided by the following exemplary description.

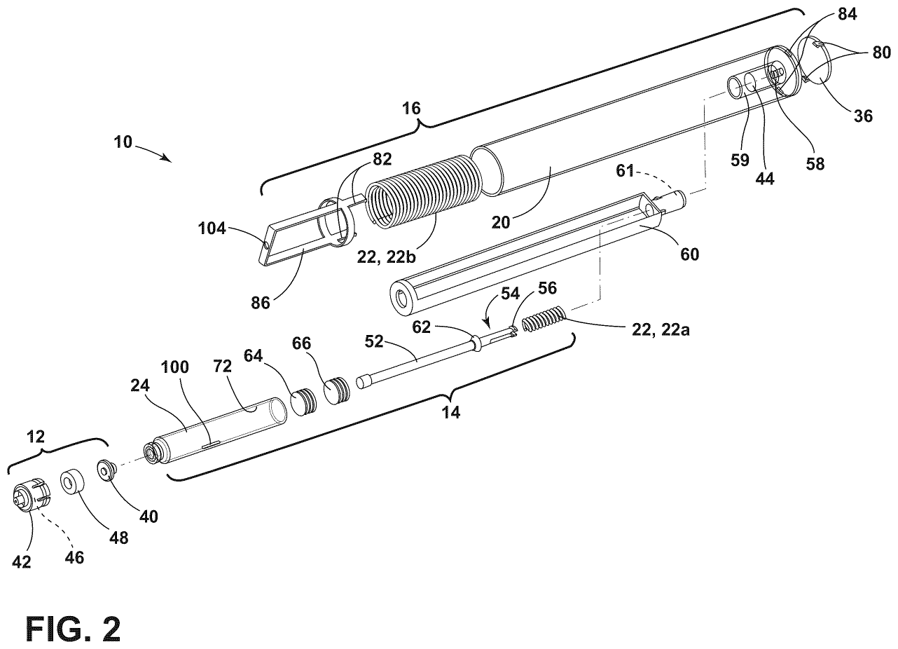

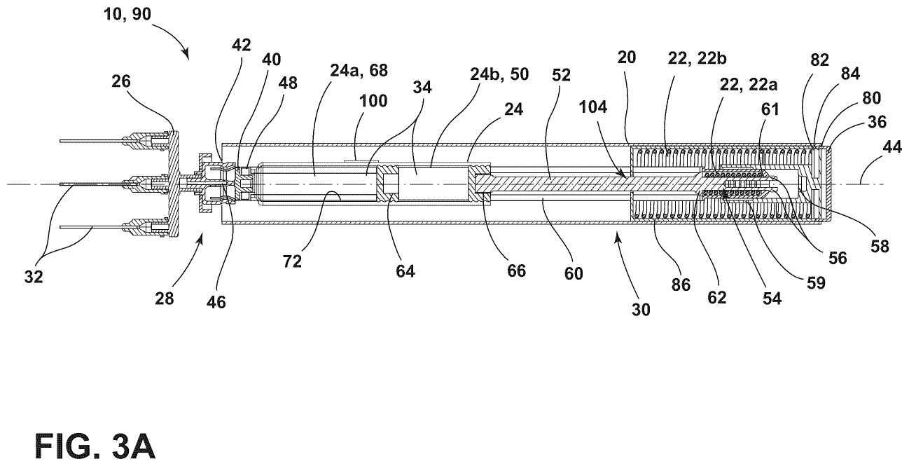

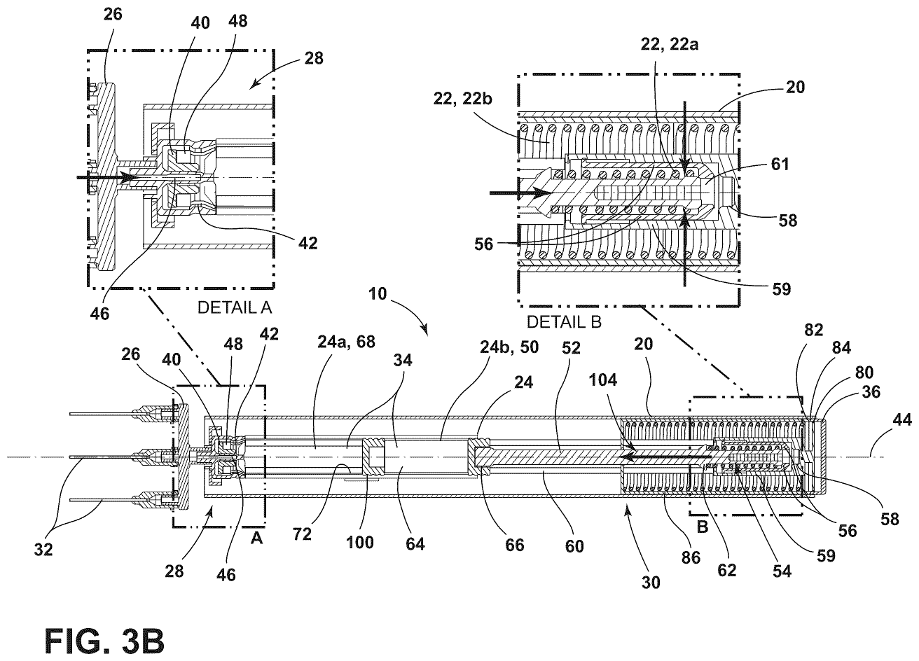

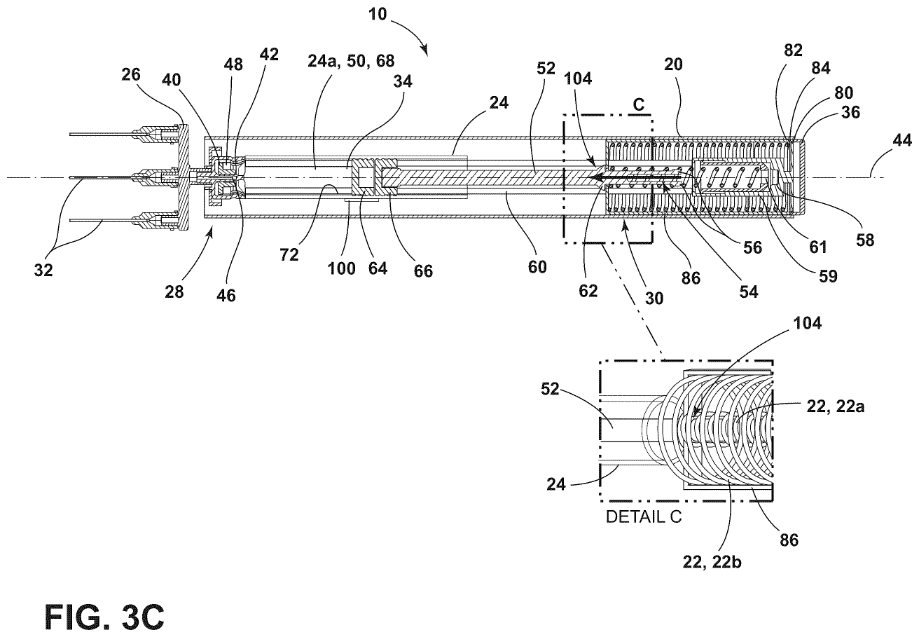

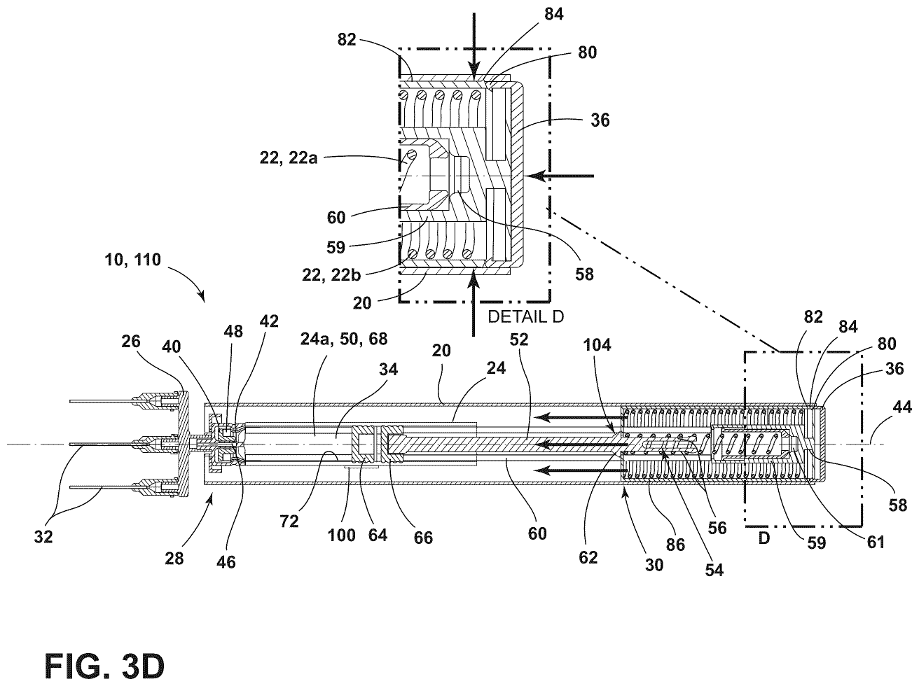

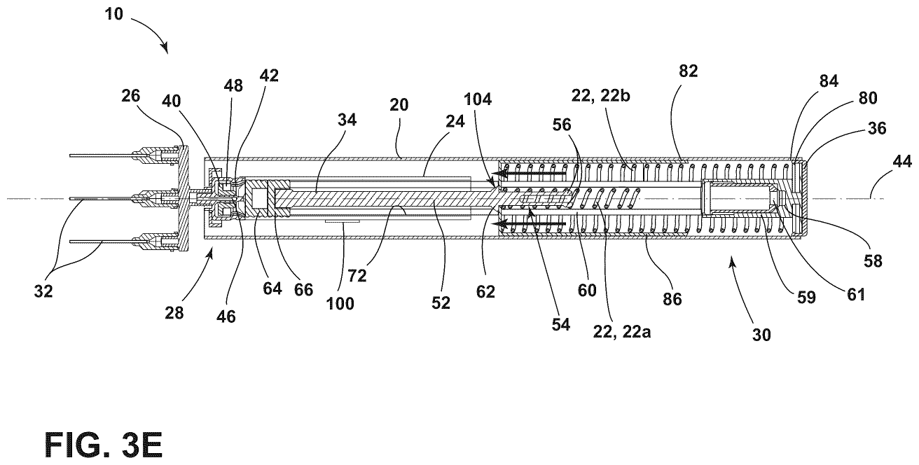

Referring to FIGS. 1A, 1B, and 2, various components and assemblies of an injection apparatus 10 are generally discussed in reference to an exemplary application. In general, the injection apparatus 10 may be formed by a plurality of subassemblies, including a seal assembly 12, a mixing assembly 14, and an injection assembly 16. Though the subassemblies of the injection apparatus 10 are separately labeled and identified, various components of the subassemblies may be commonly applied to provide the operations associated with the otherwise specifically identified subassemblies. Accordingly, the components of each of the subassemblies may provide for multiple operations that may overlap between a seal-piercing procedure, a mixing procedure, and an injection procedure. Specific examples of each of the procedures are further discussed in reference to FIG. 3A-3E.

Still referring to FIGS. 1A, 1B, and 2, the injection apparatus may be at least partially enclosed within a housing 20 which may be generally cylindrical in form to accommodate a plurality of springs 22 and a vial 24 that are similarly cylindrical in shape. The enclosure or housing 20 of injection apparatus 10 is hidden in FIG. 1B to demonstrate various components housed therein. Each of the components is further demonstrated separated in the exploded assembly view of FIG. 2.

An injection head 26 is shown in connection with an acting end portion 28 on a first side of the cylindrical body that opposes a controlling end portion 30. In operation, one or more needles 32 are connected to the injection head 26 and may be utilized to deliver an injectant 34 into body tissue (e.g., subcutaneous or intermuscular tissue). As further discussed in the following discussion, the injection of the injectant 34 may be activated by releasing a spring-loaded assembly in response to the actuation of an injection release tab 36 located on the controlling end portion 30 of the injection apparatus 10. In this way, the injectant 34 may be delivered into the tissue via the needles 32 in connection with the injection head 26 in response to an input or the application of pressure to the injection release tab 36 to quickly deliver the injectant 34 following insertion of the needles 32 into the tissue targeted for treatment.

The operation of the injection apparatus 10 may be controlled through a plurality of sequential stages that may correspond to each of the subassemblies 12, 14, and 16 as previously introduced. In a first stage, a vial seal 40 of the seal assembly 12 may be pierced or broken by a piercing cap 42 of the seal assembly. For example, if pressure is applied to the injection head 26 along a longitudinal axis 44 of the injection apparatus 10, a piercing head 46 of the piercing cap 42 may be driven through an opening formed in a crimp ring 48 of the vial seal 40, which may vent a first chamber 24a of the vial 24 to the local environment. The piercing of the vial seal 40 may allow for gas within the first chamber 24a to be displaced allowing liquid component 50 (e.g., saline solution or various solutions or injectants) stored in a second chamber 24b to enter the first chamber 24a, as later discussed.

The application of pressure to the injection head 26 along the longitudinal axis 44 may further apply pressure through the vial 24 and a plunger 52, which may release a first or mixing spring 22a of the plurality of springs 22. The mixing spring 22a may be released in response to pressure applied to a first release clip or a mixing spring release clip 54 as a result of pressure applied to a plurality of elongated prongs 56 by an actuation aperture 58. The actuation aperture 58 may be formed within an alignment sleeve 59 of the housing 20. In various implementations, the alignment sleeve 59 and the actuation aperture 58 may form components that are integral to the housing 20, as shown, or may be implemented as assembled components disposed within the housing 20. More generally, various components of the injection apparatus 10 may be combined into integral assemblies or formed by distinct assembled components without departing from the spirit of the disclosure.

When assembled, the alignment sleeve 59 may concentrically align a retention bracket 60 of the mixing assembly 14 within the housing 20. In this configuration, the elongated prongs 56 of the mixing spring clip 54 and the plunger 52 may be aligned with the actuation aperture 58 along the longitudinal axis 44. In the storage state, the elongated prongs 56 of the mixing spring clip 54 are bound within a retention aperture 61 formed in the controlling end portion 30 of the retention bracket 60. Accordingly, the interference of the elongated prongs 56 of the mixing spring clip 54 within the retention aperture 61 retain the mixing spring 20a in a compressed state. In this configuration, a length of the mixing spring 20a in the compressed state may be adjusted based on an extent of the elongated prongs 56 relative to a directionally contoured flange or directional stop 62 of the plunger 52.

As previously noted, the application of pressure to the injection head 26 along the longitudinal axis 44 may press the plunger 52 and the elongated prongs 56 of the mixing spring clip 54 against the actuation aperture 58. Accordingly, the same pressure applied to the injection head 26 along the longitudinal axis 44 that causes the piercing cap 42 to puncture the vial seal 40 may further be applied causing the vial 24 and the plunger 52 to compress the mixing spring release clip 54 against the actuation aperture 58. The pressure or pinching of the elongated prongs 56 due to the interference with the actuation aperture 58 may decouple the mixing spring release clip 54 from the retention bracket 60. Once the mixing spring release clip 54 is decoupled or unbound from the retention aperture 61 the mixing spring 22a is released from a compressed state and applies pressure to the plunger 52.

As later discussed in reference to FIG. 3A, the mixing spring 22a and the injection spring 22b are positioned concentrically within the housing 20 of the injection apparatus 10. Further, the actuation of each of the springs provides for the compound extension of the plunger 52, which is common to the operation of each of the springs 22. That is, the extension of the mixing spring 22a forces the plunger 52 forward and locks the plunger 52 to the plunger collar 86 via the directionally contoured flange or directional stop 62. Once locked to the plunger collar 86, the force of the injection spring 22b is applied from the plunger collar 86 through the same plunger 52 to administer the injectant 34. This compound operation of the concentric springs 22 by engaging the common plunger 52 provides for the injection apparatus 10 to maintain a modest package size and utilize common components to achieve the actuation driven by each of the springs 22.

The release of the mixing spring 22a may result in its expansion along the longitudinal axis 44, which applies pressure to a directional stop 62 of the plunger 52. As discussed later in reference to FIGS. 3B and 3C, the extension of the mixing spring 22a may result in the plunger 52 driving a first plunger seal 64 and a second plunger seal 66 forward toward the injection head 26, resulting in the displacement of the liquid component 50 in the second chamber 24b into the first chamber 24a of the vial 24. The displacement of the liquid component 50 into the first chamber 24a may mix a dry component 68 with the liquid component 50, resulting in the mixing of the injectant 34. Accordingly, the displacement of the plunger 52 along the longitudinal axis 44 compresses the plunger seals 64, 66 within a barrel 72 of the vial 24, allowing the mixing assembly 14 to combine the liquid component 50 with the dry component 68 to form the injectant 34.

Once the injectant 34 is mixed, the injection apparatus 10 may be primed or prepared for an injection via an actuation of a second or injection spring 22b. The injection spring 22b may be released as a result of pressure applied to the injection release tab 36 causing spring release tabs 80 to apply pressure to compress a pair of spring retention clips 82. The pressure applied to the spring retention clips 82 causes the tabs to deform and withdraw from engagement with spring retention apertures 84 formed on an interior surface of the housing 20. Once the spring retention clips 82 are separated from the spring retention apertures 84, the injection spring 22b may expand and apply pressure to a plunger collar 86 that applies pressure to the plunger 52 along the longitudinal axis 44 via the directional stop 62 or contoured flange. Accordingly, the expansion of the injection spring 22b causes the translation of the plunger collar 86 and the plunger 52 toward the injection head 26 resulting in the displacement of the plunger seals 64, 66 and expulsion of the injectant 34 through the needles 32 of the injection head 26. In this way, the mixing and injection operations are sequentially controlled by the injection apparatus 10 by applying pressure first to the acting end portion 28 along the longitudinal axis 44 and then to the controlling end portion 30 via the injection release tab 36. A further detailed description of the exemplary operation of the injection apparatus 10 is discussed in reference to FIGS. 3A-3E.

Referring now to FIG. 3A, the injection apparatus 10 is demonstrated in a storage state 90. In the storage state 90, the vial seal 40 is intact, which prevents air from entering or exiting the first chamber 24a of the vial 24. Additionally, each of the plunger seals 64, 66 is arranged such that the liquid component 50 is isolated from the dry component 68, which may assist in the preservation of the efficacy of the injectant 34. Additionally, each of the springs 22 is positioned in a compressed configuration. That is, the mixing spring 22a is retained by the mixing spring release clip 54, and the injection spring 22b is retained by the spring retention clips 82.

Referring now to FIG. 3B, detailed views A and B demonstrate the result of force applied along the longitudinal axis 44 of the injection apparatus 10 via the acting end portion 28 or the injection head 26. The force applied to the acting end portion 28 may result in the displacement of the piercing cap 42 causing the piercing head 46 to be displaced along the longitudinal axis 44, such that the vial seal 40 is pierced as demonstrated in detail A. The pressure applied to the acting end portion 28 may further result in the vial 24 applying force along the longitudinal axis 44 to the plunger 52 via the second plunger seal 66. The displacement of the plunger 52 within the actuation aperture 58 of the retention bracket 60 is shown in detail B. As a result of the plunger 52 pressed into the actuation aperture 58, the elongated prongs 56 may be pinched together, such that the mixing spring release clip 54 from the retention aperture 61 and is free to slide forward within the retention bracket 60 and force the plunger 52 forward toward the injection head 26.

As shown in FIG. 3C, the translation of the plunger 52 along the longitudinal axis 44 toward the injection head 26 may cause both the first plunger seal 64 and the second plunger seal 66 to slide forward toward the injection head 26 within the barrel 72 of the vial 24. Though each of the plunger seals 64, 66 moves toward the injection head 26 in response to the pressure applied by the mixing spring 22a to the plunger 52, the first plunger seal 64 is only displaced a short distance until the liquid component 50 reaches a bypass valve 100. More specifically, the second plunger seal 66 may be pressed into the liquid in the second chamber 24b by pressure applied through the plunger 52. The pressure applied by the second plunger seal may be applied through the liquid through the first plunger seal 64 causing gas within the first chamber 24a to be expelled through an opening formed through the vial seal 40, which was previously punctured by the piercing cap 42. Accordingly, once the first plunger seal 64 is displaced sufficiently to provide fluid communication from the second chamber 24b to the first chamber 24a via the bypass valve 100, the first plunger seal 64 may no longer travel through the barrel 72 in response to the pressure applied by the mixing spring 22a. Instead, the pressure applied by the mixing spring 22a results in the displacement of the liquid component 50 from the second chamber 24b to the first chamber 24a via the bypass valve 100. Once the liquid component 50 is forced into the first chamber 24a as a result of the collapse of the second chamber 24b, the injection apparatus 10 may be configured in a mixing state 102. In the mixing state 102, an operator of the injection apparatus 10 may be instructed to wait or shake the injection apparatus 10 for a predetermined period of time to mix the liquid component 50 and the dry component 68 prior to actuating the injection spring 22b to deliver the injectant 34.

In addition to displacing the liquid component 50 into the first chamber 24a and providing the mixing with the dry component 68 to form the injectant 34, the actuation of the mixing spring 22a may further provide for the engagement of the directional stop 62 of the plunger 52 with a plunger retaining aperture 104 of the plunger collar 86. The directional stop 62 of the plunger 52 may correspond to a beveled flange that provides a gradual contour in a first direction and a steep boundary wall along a second direction relative to the longitudinal axis 44. Accordingly, the pressure applied by the mixing spring 22a causes the smooth contoured edge of the directional stop 62 to pass through the plunger retaining aperture 104 of the plunger collar 86. Once engaged with the plunger retaining aperture 104, the steep trailing wall of the directional stop 62 may bind and lock the plunger 52 in an extended position extending outward toward the injection head 26. The binding or locking of the directional stop 62 to the retaining aperture 104 of the plunger collar 86 is demonstrated in detail C of FIG. 3C. In this configuration, longitudinal force applied to the plunger collar 86 by the injection spring 22b is transferred along the longitudinal axis 44 into the plunger 52 to provide an injection force causing the expulsion of the injectant 34 from the first chamber 24a.

Referring now to FIGS. 3D and 3E, an injecting procedure of the injection apparatus 10 is described in reference to the injection state 110 as shown. As previously discussed, the application of force along the longitudinal axis 44 to the injection release tab 36 results in the release tabs 80 compressing the retention clips 82 of the plunger collar 86, such that the retention clips 82 are released from binding within the spring retention apertures 84 of the housing 20. Once the retention clips 82 are displaced from the spring retention apertures 84, the injection spring 22b is released and expands along the longitudinal axis 44. The expansion of the injection spring 22b within the housing 20 applies pressure to the plunger collar 86 and the plunger 52. A detailed depiction of the interaction between the spring retention clips 82 and the spring release tabs 80 is shown in detail D of FIG. 3D.

As shown in FIG. 3E, the pressure of the injection spring 22b is applied through the plunger 52, which causes both the first plunger seal 64 and the second plunger seal 66 to pressurize and expel the injectant 34 from the first chamber 24a outward through the needles 32 of the injection head 26. As previously discussed, at this stage, the injectant 34 includes both the liquid component 50 and the dry component 68. Accordingly, the actuation of the injection release tab 36 may be completed after the needles 32 are inserted into the tissue, such that the injectant 34 can be delivered into the tissue as a result of the force applied by the injection spring 22b. Though various components are discussed in this application and provide for multiple purposes, it shall be understood that the components may be combined and separated to suit various applications without departing from the spirit of the disclosure.

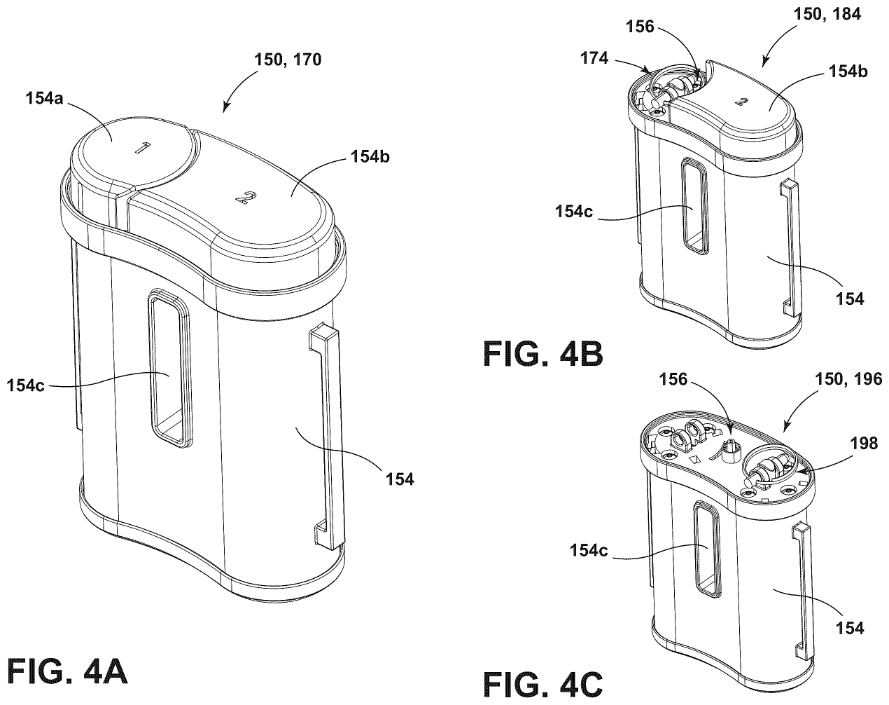

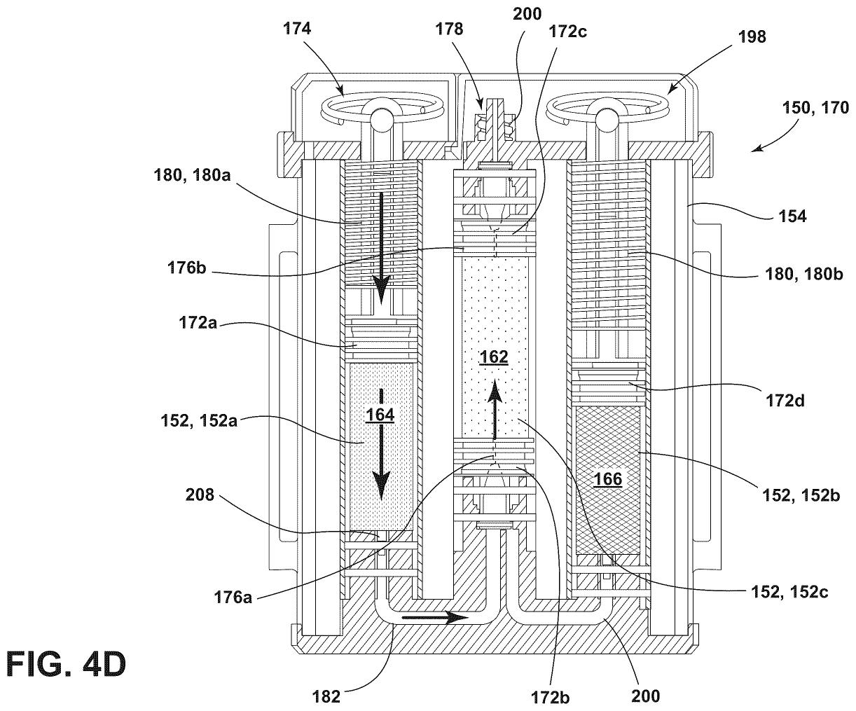

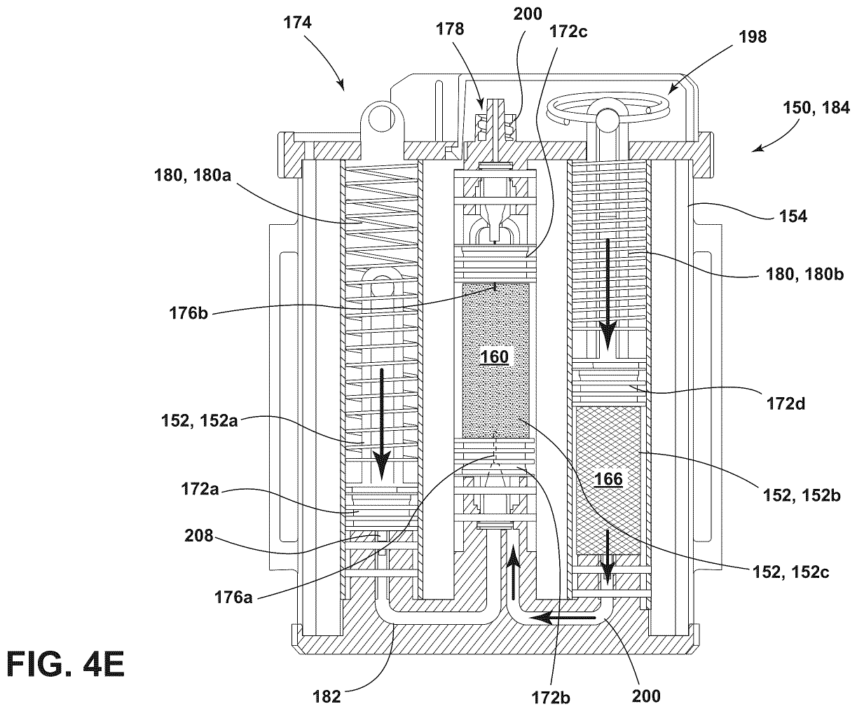

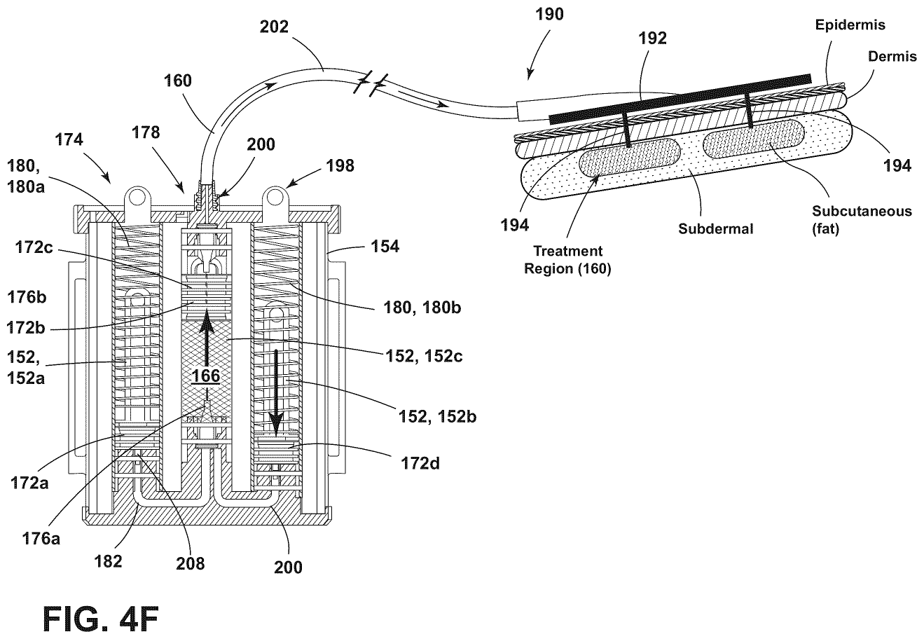

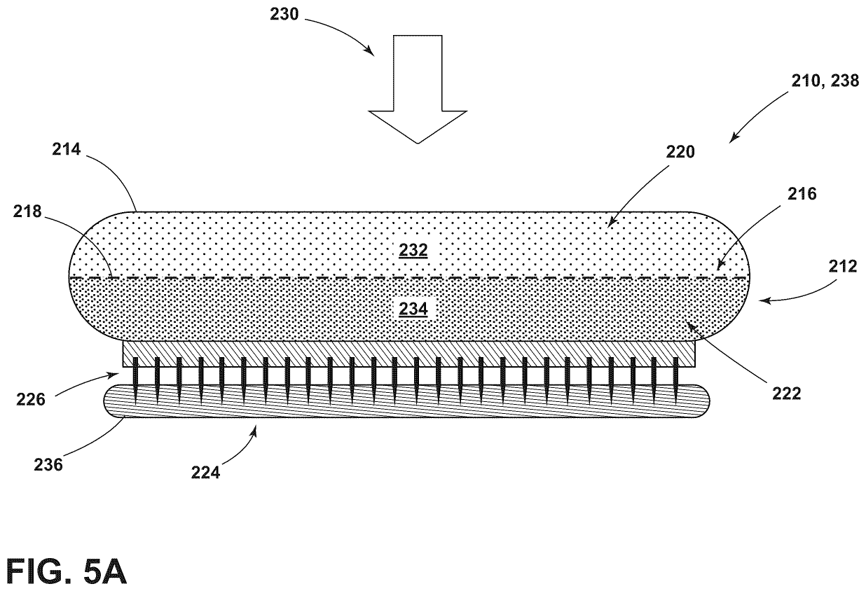

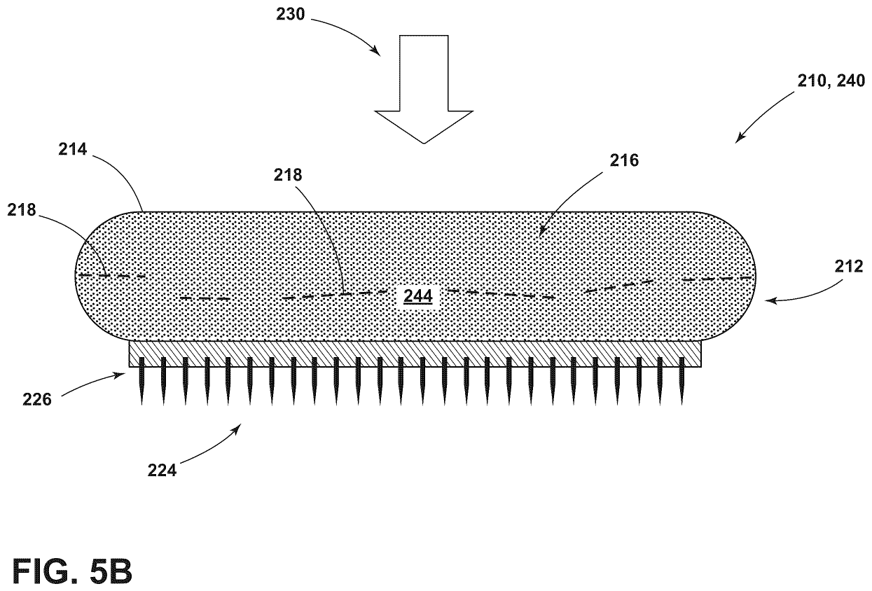

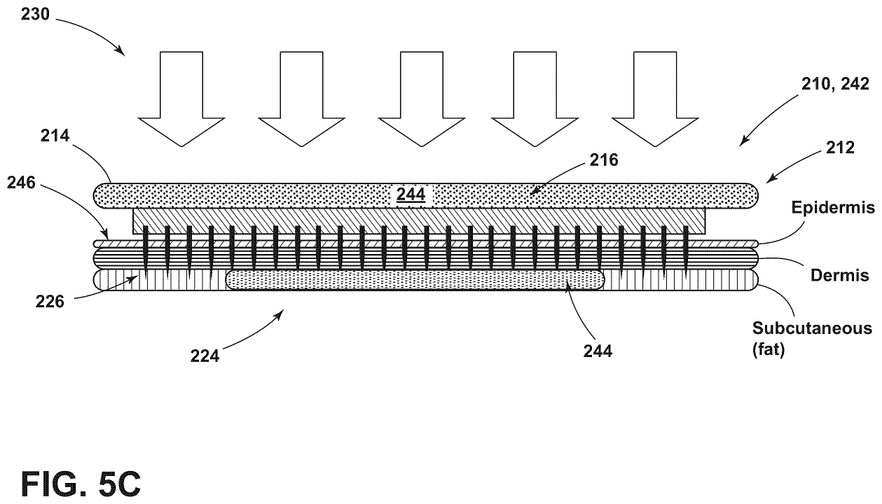

As further discussed in reference to FIGS. 4A-5D, the injection apparatus 10 may be implemented in a variety of configurations to suit packaging and performance constraints that may vary for a variety of applications. For example, as demonstrated in FIGS. 4A-4F, an injection apparatus 150 may be implemented similar to the injection apparatus 10 but may differ in that a plurality of chambers 152a, 152b, 152c may be arranged in an adjacent or parallel configuration rather than the longitudinally aligned configuration of the injection apparatus 10. Additionally, in some implementations, one or more of the plurality of chambers 152 may be aligned longitudinally. In the example demonstrated in FIGS. 4A-4F, the plurality of chambers 152 comprise a first chamber 152a, a second chamber 152b, and a third chamber 152c. Each of the plurality of chambers 152 is longitudinally aligned within a housing 154 along an output direction that is oriented perpendicular to an interface surface 156. Accordingly, the corresponding features of the injection apparatus 10 and the injection apparatus 150 may be combined in a variety of ways to suit a desired application.

Referring first to FIGS. 4A-4C, the injection apparatus 150 may provide for a plurality of configurations including a storage configuration 170 (FIG. 4A), a mixing configuration 184 (FIGS. 4B and 4D), and an injection configuration 196 (FIGS. 4C, 4E, and 4F). In each of the configurations, the housing 154 may provide for one or more features that may provide instructions and/or prevent unintended use of the apparatus 150 that could result in failure to effectively deliver an injection treatment. Such features may be particularly beneficial in cases where urgent attention is needed. As best shown in FIG. 4A, the housing may include a plurality of access covers 154a, 154b, which may correspond to caps or enclosures that limit access to a first retention device 174 and a second retention device 198. As shown, the access covers 154a, 154b may enclose the interface surface 156 and include numbers, symbols, and/or instructions identifying an order of the mixing and injection procedures required to administer a treatment with the apparatus. Further, the housing 154 may include a viewing window 154c (e.g., a transparent panel or surface) that may allow a user to view the third chamber 152c to visibly verify successful mixing of a lyophilized medication or dry component 162 and a diluent or liquid component 164. The operation of the apparatus 150, including the mixing and injecting steps required to administer a treatment, is further described in reference to FIGS. 4D-4F.

Referring now to FIGS. 4D-4F, similar to the injection apparatus 10, the injection apparatus 150 may provide for mechanically assisted mixing and administration of a dissolved solution 160 (see FIG. 4D) formed from a lyophilized medication or dry component 162 and a diluent or liquid component 164. The injection apparatus 150 may provide for the sealed and separated storage of the dry component 162 and the liquid component 164 to prevent expiration of one or more active ingredients or perishable components of the resulting dissolved solution 160. As shown, the liquid component 164 is stored in the first chamber 152a, and the dry component 162 is stored in the third chamber 152c. The second chamber 152b may be provided to assist in the injection of the dissolved solution 160 and may contain a hydraulic fluid 166 (e.g., an inert, incompressible liquid) that may be forced into the third chamber 152c to displace the dissolved solution 160.

Still referring to FIGS. 4D-4F and particularly in reference to FIG. 4D, in a resting or storage configuration 170, the chambers 152 may be separated by one or more plungers 172. The plungers 172 may comprise a first plunger 172a disposed in the first chamber 152a. In operation, the first plunger 172a may be displaced through a barrel of the first chamber in response to the release of the first retention device 174 (e.g., a pin, detent, or latch). The actuation or release of the first retention device 174 may release one of a plurality of springs 180, denoted as a mixing spring 180a, which may force the liquid component 164 or diluent through a valved exchange passage 182, through a second plunger 172b, and into the third chamber 152c or the mixing chamber. As shown by hidden lines, the valved exchange passage 182 may be fluidically connected to the third chamber 152c via a first needle 176a that extends through the body of the second plunger 172b.

The valved exchange passage 182 may comprise a valve 208, which may correspond to a spring-loaded back flow preventer or check valve that allows the liquid component 164 to be directionally passed from the first chamber 152a into the third chamber 152c. In operation, the valve 208 may prevent backflow from the third chamber 152c to the first chamber 152a. This valve 208 may prevent aqueous liquid of the liquid component 164 in the chamber 152a from prematurely mixing with the dry component 162 (e.g., lyophilized, dried medicament) stored in the third chamber 152c. Additionally, the valve 208 may prevent any backwards flow of the hydraulic fluid 166 in the third chamber 152c from flowing into the first chamber 152a once the injection spring 180b is actuated and the second plunger 172b has moved off the first needle 176a.

As later discussed in reference to the injection configuration 196, the pressure of the hydraulic fluid 166 applied to the second plunger 172b may cause the second plunger 172b to traverse the third chamber 152c, causing the first needle 176a to be withdrawn from the second plunger 172b. The aperture or hole formed by the first needle 176a may seal via an adhesive, sealing compound, or by a compression of an elastomeric material into the passage formerly occupied by the first needle 176a. In this way, the second plunger 172b may allow for the liquid component 164 to enter the third chamber 152c in the mixing configuration 170 and also separate the hydraulic fluid 166 from the dissolved solution 160 in the injection configuration 196. The entry of the liquid component 164 into the third chamber 152c may cause the dry component 162 to be combined with the liquid component 164 to form the dissolved solution 160.

Upon extension of the mixing spring 180a, the dissolved solution 160 may occupy the third chamber 152c formed by the combination of the liquid component 164 with the dry component 162. Prior to the release of the injection spring 180b, a third plunger 172c may retain the solution 160 in the third chamber 152c proximal to a fluid outlet port. Once the solution 160 is dissolved in the third chamber 152c, the second cap 154b may be removed and the apparatus 150 may be prepared for injection. The release of the second retention device 198 (e.g., pin), may result in the hydraulic fluid 166 applying the spring force of the injection spring 180b on the second plunger 172b. The hydraulic fluid 166 may displace the second plunger 172b and compress the dissolved solution 160, thereby forcing the third plunger 172c into a second needle 176b. The pressure applied to the third plunger may cause the second needle 176b to puncture the third plunger 172c and fluidically couple the third chamber 152c with the fluid outlet port 178. As later discussed in reference to FIG. 4F, the injection configuration 196 may further require the attachment of a syringe, needle set, or injector assembly 190, which may comprise a reservoir 192 fluidically coupled to an injection needle set 194.

Referring primarily to FIG. 4E, the injection apparatus 150 is shown in the injection configuration 196. Note that the injector assembly 190 is omitted from FIG. 4E and demonstrated in FIG. 4F for clarity. Similar to the liquid component 164, the hydraulic fluid 166 may be delivered into the third chamber 152c via an exchange passage 200. In operation, the withdrawal of the second retention device 198 may result in the application of an injection force of the injection spring 196 on a fourth plunger 172d and the hydraulic fluid 166, pressing the hydraulic fluid against the second plunger 172b. As previously discussed, the pressure of the hydraulic fluid 166 applied to the second plunger 172b may cause the second plunger 172b to traverse the third chamber 152c, causing the first needle 176a to be withdrawn from the second plunger 172b. The aperture or hole formed by the first needle 176a may seal via an adhesive, sealing compound, or by a compression of an elastomeric material into the passage formerly occupied by the first needle 176a. In this way, the pressure of the hydraulic fluid 166 may be applied to the second plunger 172b, which may separate the hydraulic fluid 166 from the dissolved solution 160 in the third chamber 152c. The displacement of the second plunger 172b in the third chamber 152c may result in the pressure of the hydraulic fluid 166 being applied to the dissolved solution 160, thereby forcing the third plunger 172c into a second needle 176b. Accordingly, the mixing and injection of the dissolved solution 160 may be mechanically assisted. In general, the first and second needles 176a, 176b may correspond to barbs, needles, pins, lumens, or various devices that may rupture or otherwise break a seal formed by the plungers 172b, 172c. These rupturing devices are generally referred to as needles 176 and may allow for the passage of fluid by creating a passage or cannula in configurations where the seal associated with the plungers 172b, 172c are ruptured or otherwise breached. Accordingly, though referred to as needles 176 (e.g., lure lock needles, syringes, cannulas, etc.), the rupturing devices may be implemented in by a variety of sharp or narrow devices that may provide for penetration through a portion of the plungers 172b, 172c and the passage of the liquid component 164 and the dissolved solution 160, respectively.

Referring now to FIG. 4F, the injection apparatus 150 is shown in connection with the injector assembly 190 via a fitting 200 (e.g., compression, threaded, etc.) at the fluid outlet port 178. As shown, the which may comprise a reservoir 192 fluidically coupled to an injection needle set 194 or plurality of injection needles 194. The injector assembly 190 may include an elongated tube 202 fluidically coupling the fluid outlet port 178 with the injection staging injection staging reservoir 192. Accordingly, the combined assembly 150 in the injection configuration 196 may operate as provided in the following exemplary steps. The extension of the injection spring 180b may force the fourth plunger 172d through the barrel of the second chamber 152b, thereby displacing the hydraulic fluid 166 into the third chamber 152c via an exchange passage 200. In response to the entry of the hydraulic fluid 166 into the third chamber 152c, the second plunger 172b may be forced through the barrel of the third chamber 152c. In response to the displacement of the third plunger 172c, the dissolved solution 160 or injectant may be forced through the opening formed in the second plunger 172b by the second needle 176b. The dissolved solution 160 may then be forced through the fluid outlet port 178, into the injection staging reservoir 190 where the dissolved solution 160 is distributed among the injection needle set 194 into the target site. Once the injection spring 180b is fully extended, the injection process may be completed and the injection apparatus 150 may be spent.

As previously discussed, the first and third chambers 152a, 152c may be separated by the valved exchange passage 182. As demonstrated in the steps shown in FIGS. 4D-4F, the valved exchange passage 182 may comprise the valve 208 (e.g., backflow preventer valves, check valves, directional valves, or etc.) that may prevent the backflow of the dissolved solution 160 from returning from the third chamber 152c into the first chamber 152a via the exchange passage 182. By preventing the backflow of the dissolved solution into the first chamber 152a, the injection apparatus 150 may ensure that the hydraulic pressure created by the displacement of the fourth plunger 172d is not diminished or dampened as a result of fluid in the third chamber 152c passing into the first chamber 152a and compressing the mixing spring 180a.

As generally demonstrated in FIGS. 4A-4F, the injection apparatus 150 may provide for a wider package profile than the elongated assembly of the injection apparatus 10. In various implementations, the housing 154 may be enclosed within a protective shroud or casing, which may structurally reinforce the injection apparatus 150 while also preventing the unintentional release of the mixing spring 180a and/or the injection spring 180b. As shown, the injection apparatus 150 may extend longitudinally parallel to the plurality of springs 180 and perpendicular to the interface surface 156, having a substantially front rectangular profile. As depicted in FIGS. 4A-4C, the top profile may correspond to an elongated ovular, bean, or elliptical shape, which may effectively house the plurality of chambers 152. Though illustrated as an ovular shape, the top profile may conform to a variety of shapes to provide for the housing 154 to enclose or surround the chambers 152.

Referring now to FIGS. 5A-5D, an injection apparatus 210 is demonstrated in the form of an injection patch 212. The injection patch 212 may comprise a pouch 214 forming a sealed interior volume 216. As shown, the interior volume 216 may be separated by a puncturable membrane 218 into a first chamber 220 and a second chamber 222. As demonstrated in FIG. 5D, the injection patch 212 may form an injection surface 224 that may be substantially elliptical, ovular, rectangular, circular, or conform to various other profile shapes. In general, the shape of the injection patch may be configured to extend over an injection site for administration of a liquid medication or injectant over an injection treatment area. In various implementations, the injection patch 212, including the first chamber 220 and the second chamber 222, may extend approximately coextensive over the injection surface 224. The injection surface 224 may comprise a microneedle array 226 aligned perpendicular to the injection surface in an injection direction denoted by arrows 230, as shown in FIG. 5A.

In the exemplary embodiment shown, the second chamber 222 may be interposed between the first chamber 220 and the injection surface 224 of the injection patch 212. In such configurations, a dry component 162 or lyophilized medication or drug may be enclosed within the first chamber 220, and the liquid component 164 or liquid diluent may be enclosed within the second chamber 222. Note that in some cases the liquid component may also contain a medication or treatment that may be associated with the dry component 162 or provide independent clinical benefits (e.g., a numbing agent). As previously discussed, the first chamber 220 and the second chamber 222 may be separated by a puncturable membrane 218. Additionally, the microneedle array 226 may be enclosed by a retention pad 236 or blocked from the second chamber 222 by a removable seal (not shown). The retention pad 236 or removable seal may prevent the liquid component 234 from being ejected outward through the microneedle array 226 prematurely or before the injection apparatus 210 is prepared for injection. Accordingly, the injection apparatus 210 in the form of the injection patch 212 may similarly provide for the separated storage of a two-part injection component to prevent aging and/or expiration of a medication.

Still referring to FIGS. 5A-5C, the injection apparatus 210 is demonstrated in a storage configuration 238, mixing configuration 240, and injection configuration 242, respectively. From the storage configuration, the injection patch 210 may be squeezed, bent, and/or twisted (e.g., similar to a cold pouch), such that the puncturable membrane 218 is ruptured, thereby connecting and/or merging the sealed interior volume 216 formed by the first chamber 220 and the second chamber 222. With the puncturable or intermediate membrane 218 no longer separating the dry component 232 from the liquid component 234, the injection patch 212 may be massaged, bent, flexed or otherwise manipulated, such that the liquid component 234 and the dry component 232 are thoroughly mixed to form a dissolved solution 244. Accordingly, the materials associated with the injection patch 212 forming the sealed interior volume 216 may correspond to flexible, puncture-resistant materials, such as plastics, rubberized materials, silicone, or similar materials that may retain the dissolved solution 244 throughout a manual mixing process. Similarly, the retention pad 236 may correspond to a flexible or a malleable adhesive that may prevent leakage of the dissolved solution 244 through the microneedle array 226 throughout the manipulation of the injection patch 212 to effectuate the mixing of the dissolved solution 244. In some cases, in order to clearly identify that the dry component 232 and the liquid component 234 are effectively mixed, the dissolved solution 244 may have a characteristic appearance which may be enabled by a blending of one or more of the dry component 232 and the liquid component 234. For example, the combined dissolved solution 244 may be a different color than the constituent parts when separated in the first chamber 220 and the second chamber 222.

Once the dissolved solution 244 is effectively mixed within the sealed interior volume 216, the retention pad 236 may be peeled away or removed from the microneedle array 226. As previously discussed, the retention pad 236 may correspond to a flexible polymeric or adhesive cover that may be peeled away from the microneedle array 226 in order to allow the dissolved solution 244 to flow therethrough. With the retention pad 236 removed, the injection surface 224 may be aligned over a target area and the microneedle array 226 may be pressed firmly against the tissue intended for treatment. As shown in FIG. 5C, the microneedle array 226 may extend dermally, subdermally, subcutaneously, and/or to an intramuscular injection depth, such that the dissolved solution 244 may be injected into the target site by applying pressure to an application surface 246 of the injection patch 212.

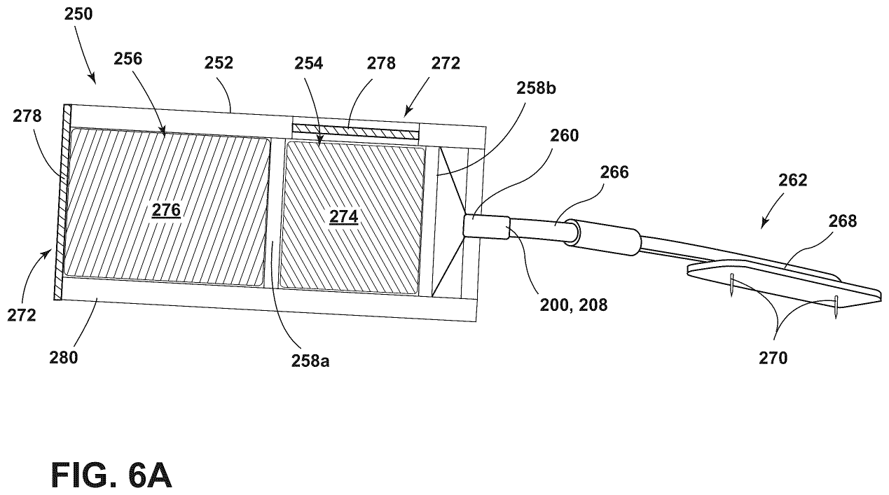

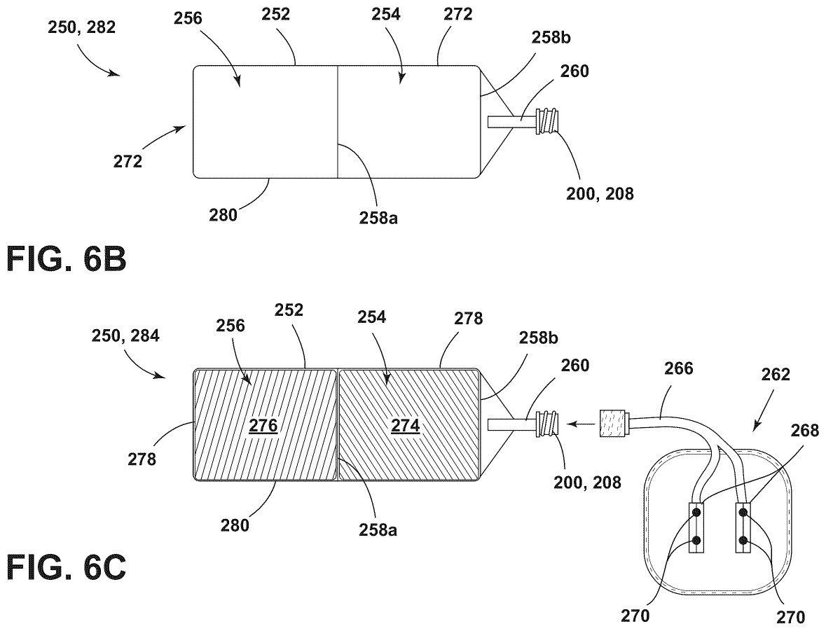

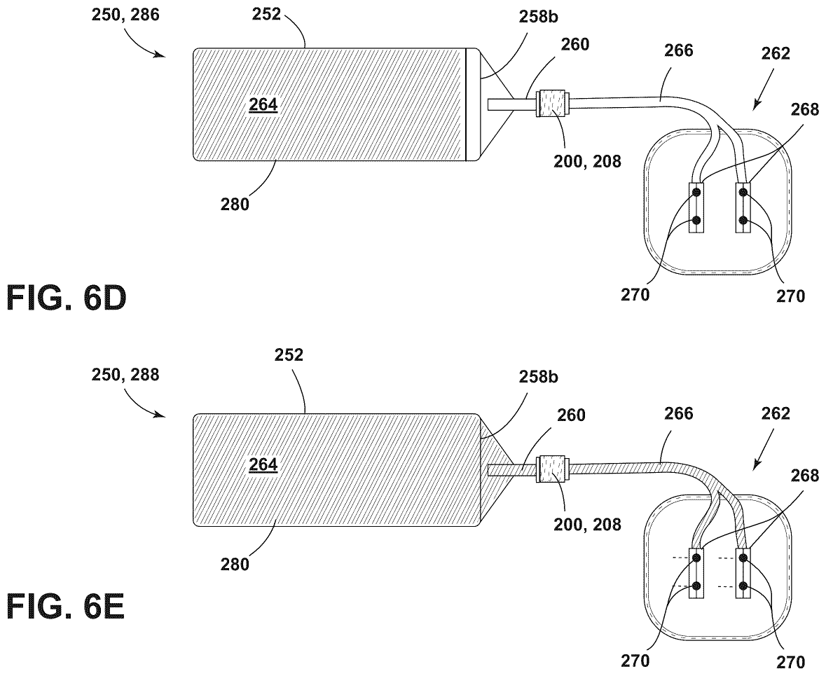

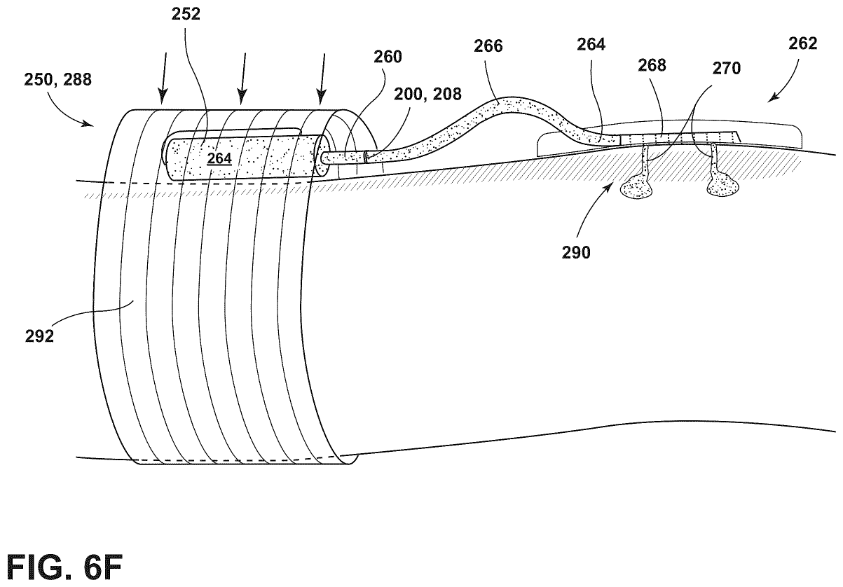

In order to sustain the force applied to the application surface 246 of the injection patch 212, in some implementations, the injection apparatus 210 may be provided with or connected with a compression device, flexible wrap or bandage (e.g., an Ace bandage, retention strap, or similar device). In such implementations, following the application of the microneedle array 226 to the target area, the compression device or retention strap may be tightened over the entirety of the injection patch 212 corresponding to the surface area over which the injection surface 224 supplies the dissolved solution 244 to the target area. The retention device, wrap, strap, etc. may then be secured to an appendage or portion of the treated individual, which may allow for continuous delivery of the dissolved solution 244 even in cases where the patient loses consciousness or is forced to withdraw pressure from the injection patch 212. An example of a compression device applied in a similar configuration is described later in reference to FIG. 6F.

Referring now to FIGS. 6A-6F, yet another implementation of the injection apparatus 250 is shown. Similar to the injection apparatus 210, the apparatus 250 may comprise to a soft flexible package design that may require additional manual steps while also limiting manufacturing costs. Referring first to the projected view in FIG. 6A, the injection apparatus 250 may comprise a segmented or subdivided pouch 252 comprising a first chamber 254 and a second chamber 256 separated by a first puncturable or frangible membrane 258a. Similar to the injection apparatus 150, the pouch may comprise a fluid outlet port 260 that may be selectively attached to an injector assembly 262 via a fitting 200 (e.g., compression, threaded, etc.). A second frangible membrane 258b may seal the second chamber 256 to prevent contamination via the outlet port 260 or premature ejection of the dissolved solution 264. In this configuration, a dissolved solution 264 may similarly be delivered via an elongated tube 266 to a reservoir 268 to deliver a treatment to a plurality of injection needles 270.

As shown in FIG. 6A, the first chamber 254 and the second chamber 256 may comprise fill openings 272. The fill openings 272 may provide for the chambers 254, 256 to be accessible to add or supply a dry component 274 or lyophilized medication and a liquid component 276 or liquid diluent in the chambers 254, 256. Once the dry component 274 and the liquid component 276 are disposed in the separate chambers 254, 256, the fill openings 272 may be closed and sealed via a perimeter seal 278. For example, the perimeter seals 278 may be provided by a heat-seal, adhesive, weld, or other processes that may enclose a perimeter 280 of the pouch 252. Once the apparatus 250 is prepared with the dry component 274 and the liquid component 276, the apparatus 250 may be stored for use. A method associated with the use of the apparatus 250 is discussed in reference to FIGS. 6B-6F in the following description.

As shown in FIG. 6B, the injection apparatus 250 is shown prior to the filling of the chambers 254, 256 with the dry component 274 and the liquid component 276. The configuration of FIG. 6B may correspond to an initial manufactured state or pre-charge state 282, which may allow the apparatus 250 to be shipped to destinations at minimal expense and allow regional treatments to be stored in the chambers 254, 256. In FIG. 6C, the chambers 254 and 256 are filled with the dry component 274 and the liquid component 276 and the perimeter 280 may be fluidically sealed via the perimeter seals 278 to provide a charged configuration 284. Accordingly, FIG. 6C may represent the state or condition that the apparatus 250 would be accessed in the field or for practical applications.

Referring still to FIG. 6C, typical use of the apparatus 250 may begin by connecting the outlet port 260 to the injector assembly 262 via the fitting 200. However, in some implementations, the connection of the outlet port 260 to the injector assembly 262 may be a final step prior to delivery of the dissolved solution 264. In such cases, the attachment of the injector assembly 262 to the fitting 200 may puncture a seal at the outlet port 260, resulting in the discharge of the dissolved solution 264. Additionally, the injector assembly 262 may be applied to the port 260 in an initial state and the reservoir 268 of the injector assembly 262 may be filled with saline prior to injection to prevent injection of air. Referring back to the example depicted, following the attachment of the injector assembly 262 to the fitting 200 in FIG. 6D, the first frangible membrane 258a may be broken by a user by squeezing the first chamber 254 of the subdivided pouch 252. As shown, the result of the breakage of the first frangible membrane 258a may allow the user to massage or knead the pouch 252, such that the dissolved solution 264 is formed by the combined dry component 274 and liquid component 276.

In various implementations, the fitting 200 may include the valve 208 as previously discussed to prevent backflow from the injector assembly 262 into the pouch 252. The valve may improve operation to control the flow of the dissolved solution 264 and/or saline. In some cases, the valve 208 may prevent backflow of the dissolved solution 264 as a result of intercompartment pressures that may build up in the patient due to swelling or inflammation. Accordingly, the inclusion of the valve 208 in the form of a backflow preventer may allow the injection apparatus 250 to be implemented in cases where the injection process may not be consistently monitored in the field.

Once the dissolved solution 264 is distributed through the merged first and second chambers 254, 256, the injection apparatus 250 may be arranged in a pre-injection configuration 286. At this stage, the user may break the second frangible membrane 258b to prepare the apparatus 250 in an injection configuration 288 as shown in FIG. 6E. In this configuration, the injector assembly 262, including the elongated tube 266, the reservoir 268, and the injection needles 270, may be primed with the dissolved solution 264, and the needles 270 may be attached to an affected region 290 as shown in FIG. 6F. As shown in FIG. 6F, the injection needles 270 may penetrate the treatment region 290 to deliver the dissolved solution 264 to the treatment region 290. For example, the injection needles 270 or needle set may be configured to deliver the dissolved solution 264 as an injectant dermally, subdermally, subcutaneously, and/or to an intramuscular injection depth. With the injection needles 270 engaged with the treatment region, the pouch 252 and/or the injector assembly 262 may be affixed to a patient via a compressive device 292, which may be in the form of a flexible bandage, strap, belt, cloth wrap, etc., that may retain the injection apparatus 250 in connection with the patient while applying pressure that may be necessary to slowly deliver or administer the dissolved solution 264 to the treatment region 290.

The disclosed examples of the injection apparatus discussed herein may provide for a variety of solutions to store, mix, and deliver a multi-part medication for field use. Though each of the implementations of the injection apparatus 10, 150, 210, and 250 are described separately, it shall be understood that many of the features may be implemented interchangeably or in combination to suit a variety of applications. Therefore, the specific scope of the inventive subject matter discussed herein shall be interpreted in light of the claims and not based on the individual teachings of the detailed description.

According to some aspects of the disclosure, an injection apparatus for administering an injectant comprises a plurality of chambers. The chambers include a mixing chamber having a first impermeable membrane disposed therein, a first chamber containing a dry component, and a second chamber containing a liquid component. The dry component and the liquid component combine in the mixing chamber to form a liquid injection solution. The mixing chamber is configured to retain the liquid injection solution formed by a combination of the dry component and the liquid component. The injection apparatus is selectively configured in a plurality of states including a storage state and a mixing state. The dry component and the liquid component are separated in the storage state and merged in the mixing state. Further, the dry component is disposed within the mixing chamber in the storage state and at least one injection needle in connection with the mixing chamber. The injection needle is placed in fluid communication with the liquid injection solution in response to a rupture or penetration of a second impermeable membrane.

In accordance with various aspects, the disclosure may implement one or more of the following features or configurations in various combinations:
at least one needle is a microneedle array in fluid communication under conditions when the impermeable membrane is ruptured;
an injection staging reservoir is disposed between the mixing chamber and the microneedle array, wherein the injection staging reservoir is fluidically connected to the liquid injection solution in response to the rupture of the impermeable membrane;
the first impermeable membrane separates the liquid component from the dry component;
the first chamber is formed by a flexible pouch forming at least a portion of a perimeter seal extending about the mixing chamber;
the second impermeable membrane comprises a frangible seal formed between the mixing chamber and an injection assembly in fluid communication with the injection needle;
the liquid injection solution is output from the mixing chamber to the at least one injection needle by compressing the mixing chamber;
a puncturing mechanism configured to rupture at least one of the first impermeable membrane and the second impermeable membrane;
the rupture of the at least one of the first impermeable membrane and the second impermeable membrane is in response to a release of a spring-loaded assembly configured to displace the liquid component into the mixing chamber;
the plurality of chambers comprise barrels having a plurality of plungers that fluidically partition the barrels;
the first impermeable membrane in the mixing chamber is a first plunger of the plurality of plungers;
the first plunger separates the liquid injection solution from a hydraulic solution configured to force the liquid injection solution from the mixing chamber out through the at least one injection needle; and/or
the puncturable or flexible seal is configured to rupture in response to a deformation of the perimeter wall of the mixing chamber.

According to another aspect of the disclosure, a method for administering an injectant is provided. A dry component is provided in a first chamber, while a liquid component is provided in a second chamber. A first seal between the first chamber and the second chamber is ruptured, mixing the dry component and the liquid component in a third chamber forming the injectant by a combination of the dry component and the liquid component. The injectant is then delivered to an injector assembly comprising at least one injection needle via an outlet in connection with the third chamber. The injectant is administered via the at least one injection needle by applying pressure to the third chamber.

According to various aspects, this disclosure may implement one or more of the following features or configurations in various combinations:
breaking a second seal disposed between the third chamber and the injector assembly;
the first seal and the second seal are frangible seals broken in response to an external pressure applied to the third chamber;
the third chamber is formed by a union of the first chamber and the second chamber;
applying a compressive wrap about a portion of a patient and the third chamber, thereby administering the injectant;
the rupture of the at least one of the first seal and the second seal is in response to a release of a spring-loaded assembly configured to deliver a hydraulic fluid into the third chamber;
the rupture of the first seal results in the liquid component mixing with the dry component in response to the release of the spring-loaded assembly;
separating a hydraulic fluid from the injectant in the third chamber via a plunger; and/or
administering the injectant by releasing a spring assembly and compressing the hydraulic fluid.

According to yet another aspect of the disclosure, an injection apparatus is configured to administer an injectant. The injection apparatus comprises a first chamber containing a dry component and a second chamber containing a liquid component. A first frangible seal is disposed between the first chamber and the second chamber. A third chamber is formed by a union of the first chamber and the second chamber. A fluid outlet port is in fluid communication with at least one injection needle. A second frangible seal is disposed between the third chamber and the fluid outlet port. The first frangible seal is selectively ruptured to allow the dry component to dissolve in the liquid component and form the injectant. The second frangible seal is selectively ruptured, thereby placing the third chamber in fluid communication with the at least one injection needle.

According to a further aspect of the disclosure, an injection apparatus for administering an injectant comprises a vial forming a barrel extending along a longitudinal axis forming an acting end portion in connection with an injection head and a controlling end portion opposite the acting end portion. At least one plunger seal is disposed in the controlling end portion of the barrel and enclosing a plunger opening of the barrel. A plunger comprises an elongated body aligned with the longitudinal axis and in connection with the at least one plunger seal. The plunger comprises a flange extending radially outward from the elongated body. A first spring forms a first inside diameter enclosed about the elongated body of the plunger, wherein the first spring engages the flange of the plunger moving the plunger and the at least one plunger seal a first distance within the barrel. A plunger collar forms a retention aperture in a load bracket through which the elongated body of the plunger extends. A second spring forms a second inside diameter enclosed about the first spring and disposed within the plunger collar, wherein the second spring engages the flange via the load bracket of the plunger collar moving the plunger and the at least one plunger seal a second distance within the barrel.

According to additional aspects, the disclosure may implement one or more of the following features or configurations in various combinations:
the flange forms a directional stop comprising a sloped wall directed toward the acting end portion and a steep wall perpendicular to the longitudinal axis, wherein the steep wall directionally locks the load bracket of the plunger collar to the plunger;
a vial seal encloses the acting end portion of the vial in connection with the injection head;
the piercing cap comprising a piercing head aligned with the vial seal, wherein the piercing cap translates toward and the piercing head forms a puncture in the vial seal in response to a force applied to the acting end portion;
the second spring is concentrically arranged with the first spring about the longitudinal axis;
the second distance is applied along the longitudinal axis in addition to the first distance;
at least one plunger seal comprises a first plunger seal and a second plunger seal;
the second plunger seal is disposed in the controlling end portion of the barrel and the first plunger seal disposed in the barrel at an intermediate position between the acting end portion and the controlling end portion of the vial;
the vial forms a first chamber containing a dry component of the injectant in the barrel between the vial seal and the first plunger seal and a second chamber containing a liquid component of the injectant in the barrel between the first plunger seal and the second plunger seal;
a bypass valve formed in an intermediate section of the barrel wall proximate to the intermediate position of the first plunger seal, the bypass valve extending a bypass distance in excess of a width of the first plunger seal;
the movement of the at least one plunger seal distance over the first distance comprises moving the first plunger seal to a mixing position wherein the first plunger seal is positioned in the barrel along the bypass distance of the bypass valve;
the movement of the plunger and the at least one plunger seal distance over the first distance comprises moving the second plunger seal over the first distance adjacent to the first plunger seal;
the movement of the second plunger seal over the first distance displaces the liquid component of the injectant in the second chamber through the bypass valve and into the first chamber where the liquid component is mixed with the dry component and displaces gas in the first component out through a puncture in the vial seal;
the movement of the plunger and the at least one plunger seal over the second distance within the barrel comprises moving the first plunger seal and the second plunger seal over the second distance such that the first plunger seal and the second plunger seal are positioned in the acting end portion of the barrel;
the movement of the first plunger seal and the second plunger seal over the second distance expels the injectant comprising the dry component and the liquid component from the first chamber and through the injection head;
the second spring is retained in a compressed configuration via a plurality of spring release tabs of the plunger collar in connection with a plurality of retaining apertures formed in a housing of the injection apparatus; and/or
the second spring is released moving the plunger and the at least one plunger seal the second distance within the barrel in response to the depression of an injection release button comprising a plurality of spring release tabs that compress the release clips disconnecting the release clips from the spring retention apertures.

It will be understood that any described processes or steps within described processes may be combined with other disclosed processes or steps to form structures within the scope of the present device. The exemplary structures and processes disclosed herein are for illustrative purposes and are not to be construed as limiting.

It is also to be understood that variations and modifications can be made on the aforementioned structures and methods without departing from the concepts of the present device, and further it is to be understood that such concepts are intended to be covered by the following claims unless these claims by their language expressly state otherwise.

The above description is considered that of the illustrated embodiments only. Modifications of the device will occur to those skilled in the art and to those who make or use the device. Therefore, it is understood that the embodiments shown in the drawings and described above are merely for illustrative purposes and not intended to limit the scope of the device, which is defined by the following claims as interpreted according to the principles of patent law, including the Doctrine of Equivalents.

What is claimed is:

1. An injection apparatus for administering an injectant comprising:
a plurality of chambers comprising:
a mixing chamber having a first impermeable membrane disposed therein;
a first chamber containing a dry component; and
a second chamber containing a liquid component, wherein the dry component and the liquid component combine to form a liquid injection solution in the mixing chamber, the mixing chamber configured to retain the liquid injection solution formed by a combination of the dry component and the liquid component; and
wherein injection apparatus is selectively configured in a plurality of states comprising a storage state and a mixing state, and wherein the dry component and the liquid component are separated in the storage state and merged in the mixing state, and wherein the dry component is disposed within the mixing chamber in the storage state; and
at least one injection needle in connection with the mixing chamber, wherein the injection needle is placed in fluid communication with the liquid injection solution in response to a rupture or penetration of a second impermeable membrane.

2. The injection apparatus according to claim 1, wherein the first impermeable membrane separates the liquid component from the dry component.

3. The injection apparatus according to claim 1, wherein the first chamber is formed by a flexible pouch forming at least a portion of a perimeter seal extending about the mixing chamber.

4. The injection apparatus according to claim 1, wherein the second impermeable membrane comprises a frangible seal formed between the mixing chamber and an injection assembly in fluid communication with the injection needle.

5. The injection apparatus according to claim 1, wherein the liquid injection solution is output from the mixing chamber to the at least one injection needle by compressing the mixing chamber.

6. The injection apparatus according to claim 1, further comprising:
a puncturing mechanism configured to rupture at least one of the first impermeable membrane and the second impermeable membrane.

7. The injection apparatus according to claim 6, wherein the rupture of the at least one of the first impermeable membrane and the second impermeable membrane is in response to a release of a spring-loaded assembly configured to displace the liquid component into the mixing chamber.

8. The injection apparatus according to claim 1, wherein the plurality of chambers comprise barrels having a plurality of plungers that fluidically partition the barrels.

9. The injection apparatus according to claim 8, wherein the first impermeable membrane in the mixing chamber is a first plunger of the plurality of plungers.

10. The injection apparatus according to claim 9, wherein the first plunger separates the liquid injection solution from a hydraulic solution configured to force the liquid injection solution from the mixing chamber out through the at least one injection needle.

11. The injection apparatus according to claim 10, wherein the puncturable or flexible seal is configured to rupture in response to a deformation of the perimeter wall of the mixing chamber.

12. A method for administering an injectant comprising:
providing a dry component in a first chamber;
providing a liquid component in a second chamber;
rupturing a first seal between the first chamber and the second chamber;
mixing the dry component and the liquid component in a third chamber forming the injectant by a combination of the dry component and the liquid component;
delivering the injectant to an injector assembly comprising at least one injection needle via an outlet in connection with the third chamber; and
administering the injectant via the at least one injection needle by applying pressure to the third chamber.

13. The method according to claim 12, further comprising:
breaking a second seal disposed between the third chamber and the injector assembly.

14. The method according to claim 12, wherein the first seal and the second seal are frangible seals broken in response to an external pressure applied to the third chamber.

15. The method according to claim 12, wherein the third chamber is formed by a union of the first chamber and the second chamber.

16. The method according to claim 15, further comprising:
applying a compressive wrap about a portion of a patient and the third chamber, thereby administering the injectant.

17. The method according to claim 12, wherein the rupture of the at least one of the first seal and the second seal is in response to a release of a spring-loaded assembly configured to deliver a hydraulic fluid into the third chamber.

18. The method according to claim 17, wherein the rupture of the first seal results in the liquid component mixing with the dry component in response to the release of the spring-loaded assembly.

19. The method according to claim 12, further comprising:
separating a hydraulic fluid from the injectant in the third chamber via a plunger; and
administering the injectant by releasing a spring assembly and compressing the hydraulic fluid.

20. An injection apparatus configured to administer an injectant, the injection apparatus comprising:
a first chamber containing a dry component;
a second chamber containing a liquid component;
a first frangible seal disposed between the first chamber and the second chamber;
a third chamber formed by a union of the first chamber and the second chamber;
a fluid outlet port in fluid communication with at least one injection needle; and
a second frangible seal disposed between the third chamber and the fluid outlet port, wherein:
the first frangible seal is selectively ruptured allowing the dry component to dissolve in the liquid component forming the injectant; and
the second frangible seal is selectively ruptured thereby placing the third chamber in fluid communication with the at least one injection needle.
